(19) United States
(12) Patent Application Publication
(10) Pub. No.: US 2022/0259562 A1
(43) Pub. Date: Aug. 18, 2022

(54) COMPOSITION FOR CULTURING NATURAL KILLER CELLS, AND METHOD USING SAME

(71) Applicant: CHA UNIVERSITY INDUSTRY-ACADEMIC COOPERATION FOUNDATION, Pocheon-si (KR)

(72) Inventors: KyuBum KWACK, Seongnam-si (KR); Sooyeon OH, Seoul (KR); Hae Jong LEE, Anyang-si (KR); Jeong Min SIM, Daejeon (KR); Jae Joon LIM, Suwon-si (KR)

(73) Assignee: CHA UNIVERSITY INDUSTRY-ACADEMIC COOPERATION FOUNDATION, Pocheon-si (KR)

(21) Appl. No.: 17/610,857

(22) PCT Filed: May 14, 2020
(86) PCT No.: PCT/KR2020/006366

§ 371 Date: Nov. 12, 2021

(30) Foreign Application Priority Data
May 15, 2019 (KR) 10-2019-0057136

(51) Int. Cl. C12N 5/0783

(52) U.S. Cl. CPC C12N 5/0646; C12N 2501/998; C12N 2531/00; C12N 2501/2315

(57) ABSTRACT

Provided are a composition and a kit, each for culturing natural killer cells, and a method of using the same. According to the composition for culturing natural killer cells according to aspect, and a method of culturing natural killer cells using the same, when natural killer cells are cultured in peripheral blood mononuclear cells, they are cultured in a medium including the composition for culturing natural killer cells, the composition including magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof, thereby proliferating natural killer cells in a large quantity and promoting activation or inhibition of natural killer cells, or expansion of natural killer cells. Accordingly, the natural killer cells cultured using the same may be usefully applied to immune cell therapy products. Further, the magnetic particles may be easily separated from the medium, which is a simple and economical manner. Since the safe magnetic particles are used, they are excellent in terms of clinical safety.

### TECHNICAL FIELD

The present disclosure relates to a composition and a kit, each for culturing natural killer cells, and a method of using the same. This application is based on and claims priority to Korean Patent Application No. 10-2019-0057136, filed on May 15, 2019, in the Korean Intellectual Property Office, the disclosure of which is incorporated by reference herein in its entirety.

### BACKGROUND ART

Current general cancer therapies include surgery, radiation therapy, chemotherapy, etc., which are used alone or in combination depending on the type and stage of cancer. However, these therapies are accompanied by significant side effects and pain in patients, and the existing therapies have limitations in that they cause some damage to normal cells. Recently emerging cancer immunotherapy is a therapy, in which the human body's own immune system is utilized to more specifically remove cancer cells while minimizing damage to normal cells. Various sub-fields (antibody therapy, immune cell therapy, viral immunotherapy, nanotechnology for immunotherapy, etc.) have been actively studied. Among them, immune cell therapy is a method of treating cancer by increasing the number of natural killer (NK) cells, natural killer T cells, T cells, B cells, dendritic cells, etc. in lymphocytes obtained from a patient's blood, enhancing their functions in vitro, and then returning them to the patient's body. Such therapies using immune cells exhibit good effects in immune response-modulating treatment, and are considered to be excellent in terms of toxicity and safety.

Among the immune cells, NK cells, which are important cells responsible for innate immunity, have functions of identifying and killing abnormal cells such as virus-infected cells, tumor cells, etc. NK cells are also characterized in that they are able to recognize tumors and virus-infected cells that may not be recognized by T cells, and have superior safety, as compared with T cells. Over the last decade, tumor immunotherapy using patients' immune systems has been steadily developed, and cell therapy products using the same have been commercialized.

For the development of cell therapy products using NK cells, it is necessary to strengthen and activate functions of NK cells, and to culture and expand NK cells. Traditionally, for culturing or expanding of NK cells, a donor cell is required during culturing. A cell such as K562 is generally used as the donor cell, which is a cancer cell not suitable for clinical use.

Accordingly, it is necessary to study a method of proliferating NK cells in large quantities and a method of culturing NK cells with enhanced or suppressed activity, which are capable of solving a problem of immune rejection and providing personalized cell therapy as a cell therapy for treating intractable diseases such as cancer.

### DESCRIPTION OF EMBODIMENTS

### Technical Problem

An aspect provides a composition for culturing natural killer cells.

Another aspect provides a method of culturing natural killer cells using the composition for culturing natural killer cells.

### Solution to Problem

An aspect provides a composition for culturing natural killer cells.

The composition for culturing natural killer cells may be a composition for culturing natural killer cells, the composition including magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof.

As used herein, the term “natural killer cell (NK cell)” refers to a large granular lymphocyte (LGL) which is a type of lymphocytes, and has excellent ability to kill infected virus and tumor cells, and has a characteristic of not killing most normal cells. Thus, NK cells play an important role in the early stages of viral infection or tumorigenesis before large quantities of active cytotoxic T lymphocytes are produced. For example, when NK cells are in contact with target cells, some molecules lyse the cells by creating pores in the membrane of the target cells while other molecules enter the target cells and increase fragmentation of nuclear DNA, leading to necrosis, apoptosis, or programmed cell death.

The NK cells may be derived from, for example, a mammal, a human, a monkey, a pig, a horse, a cow, a sheep, a dog, a cat, a mouse, a rabbit, etc. The NK cells may be obtained from a normal person or a cancer patient. The NK cells may be isolated from blood or peripheral blood mononuclear cells (PBMCs). A method of isolating blood, a method of isolating PBMCs therefrom, or a method of isolating NK cells therefrom may be performed by a known method.

The composition includes magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof, thereby exhibiting improved effects of activating, proliferating, expanding, or inhibiting NK cells, as compared with a composition for culturing natural killer cells, the composition including a soluble activating receptor ligand, inhibitory receptor ligand, costimulatory receptor ligand, cytokine, cytokine receptor, immune checkpoint ligand, blocking antibody, or a combination thereof.

In the composition according to an aspect, the activating receptor ligand, which is a protein expressed in transformed cells including solid cancer cells or blood cancer cells, virus-infected cells, and stressed cells, may refer to a substance capable of inducing activation and effector function of NK cells through binding with the above-mentioned NK cell receptors. The activating receptor ligand may include ligands for natural cytotoxicity receptor (NCR) family, NKG2 family, and killer cell immunoglobulin like receptor (KIR) family, three types classified according to NK cell activating receptor structure. Further, the activating receptor ligand may be, for example, one or more selected from the group consisting of BAG6, AICL, MICA, MICB, CADM1, IgG, CD48, NTB-A/SLAMF6, CD70, CD155, CD319, C8, C9, and CS1.

The term “activating receptor ligand” may refer to a substance capable of activating a receptor by binding to a specific site of the receptor.

In the composition according to an aspect, the inhibitory receptor ligand may be, for example, one or more selected from the group consisting of HLA-A, HLA-B, HLA-BW4, HLA-C1, HLA-C2, HLA-E, HLA-G, CD112/Nectin-2, CD112/Nectin-3, cadherin, collagen, OCIL, and CLEC2D.

The term “inhibitory receptor ligand” may refer to a substance capable of inhibiting a receptor by binding to a specific site of the receptor.

In the composition according to an aspect, the costimulatory receptor ligand may include a tumor necrosis factor family (TNF family) ligand, a Toll-like receptor family (TLR family) ligand, and a virus related glycoprotein ligand. Further, the costimulatory receptor ligand may be, for example, one or more selected from the group consisting of 4-1BB ligand, CD28 ligand, NTBA, TLRL, PVR/Nectin-2, and PVR.

The term “costimulatory receptor ligand”, which is a protein/glycoprotein expressed in most nucleated cells and viruses, may refer to a ligand capable of binding to a costimulatory receptor. Further, the costimulatory receptor ligand, which is a substance mediating secondary signals, may refer to a substance capable of increasing activation and effector function of NK cells through enhancement of primary signals of NK cells, when binds with a costimulatory receptor.

In the composition according to an aspect, the cytokine may be one or more selected from the group consisting of IFN-α, IFN-β, IFN-γ, IL-1, IL-2, IL-3, IL-4, IL-6, IL-10, IL-12, IL-15, IL-17, IL-18, IL-21, and IL-27.

In the composition according to an aspect, the cytokine receptor may be IL-2Rα, IL-15Rα, or a combination thereof.

In the composition according to an aspect, the immune checkpoint ligand may include a B7 family, galectin family, and a PVR family. Further, the immune checkpoint ligand may be, for example, one or more selected from the group consisting of PD-L1, a BTLA-4 ligand, a CTLA-4 ligand (CD80), a Tim-3 ligand, IDO, A2AR, and a TIGIT ligand.

The term “immune checkpoint ligand” may refer to a protein that modulates an immune system for self-tolerance.

In the composition according to an aspect, the blocking antibody may be one or more selected from the group consisting of an anti-KIR2DL1 monoclonal antibody (mAb), an anti-KIR2DL2 mAb, an anti-KIR2DL3 mAb, an anti-KIR2DL5A mAb, an anti-KIR2DL5B mAb, an anti-KIR3DL1 mAb, an anti-KIR3DL2 mAb, an anti-KIR2DL4 mAb, an anti-CD94/NKG2A mAb, an anti-CD94/NKG2B mAb, an anti-CD96 mAb, an anti-CEACAM-1 mAb, an anti-ILT2/LILRB mAb, an anti-KLRG1 mAb, an anti-LAIR1 mAb, an anti-NKRP1A mAb, an anti-Siglec3 mAb, an anti-Siglec7 mAb, and an anti-Siglec9 mAb.

The term “blocking antibody” may refer to an immunoglobulin protein that does not react, when binds to an antigen, but prevents other existing antibodies from binding to the antigen. Specifically, the blocking antibody is a Y-shaped protein consisting of two light chains and two heavy chains by disulfide bonds, and has a constant region and a variable region. Depending on a difference in the constant region of the heavy chain, the antibody may have IgA, IgD, IgM, IgE, and IgG isotypes, and may inhibit binding between multiple proteins through modification of an amino acid sequence in the variable region. The above-mentioned blocking antibody may be for a receptor/ligand that plays a role in activating, inhibiting, co-stimulating, and immune checkpoint for NK cells.

In the composition according to one aspect, at least one surface of the magnetic particles may be coated with protein G or protein A. The protein G or protein A has high binding affinity to immunoglobulins, and the type thereof is not limited as long as it may be coated on the magnetic particles.

In the composition according to an aspect, the activating receptor ligand, the inhibitory receptor ligand, the costimulatory receptor ligand, the cytokine, the cytokine receptor, the immune checkpoint ligand, and the blocking antibody may be in a fusion form with a human immunoglobulin.

In the composition according to an aspect, the IL-15R may be interleukin-15 receptor α (IL-15Rα).

The IL-15R may be an IL-15 receptor expressed in NK cells, and may be involved in enhancing growth and differentiation of NK cells. Among the receptors, for example, IL-15Rα may perform not only classical signaling, but also trans-signaling. The trans-signaling may refer to transmission of activation signals due to trans-crosslinking between cells, even though cells do not express IL-15 Ra, when IL-15 Ra is expressed on the surface of a neighboring cell and IL-15 binds to the receptor. As described above, a biological mechanism of receptors capable of trans-signaling may be applied to the composition for culturing according to an aspect.

The 4-1BB ligand is known as a molecule involved in the expansion of NK cells.

In the composition according to an aspect, the human immunoglobulin may be a human immunoglobulin G.

In the present disclosure, the magnetic particle may include any one, as long as it is a particle having magnetism. Specifically, the magnetic particle may include one or more magnetic elements selected from the group consisting of iron (Fe), nickel (Ni), cobalt (Co), manganese (Mn), bismuth (Bi), zinc (Zn), strontium (Sr), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), ruthenium (Lu), copper (Cu), silver (Ag), gold (Au), cadmium (Cd), mercury (Hg), aluminum (Al), gallium (Ga), indium (In), thallium (TI), calcium (Ca), barium (Ba), radium (Ra), platinum (Pt), and lead (Pd). The magnetic particle may be oxidized or surface-modified. Specifically, it may be modified with protein G or protein A. By the modification, the binding affinity of the magnetic particle to immunoglobulin may be improved.

The magnetic particles may be used after being prepared by a known method, or may be used after being commercially purchased.

The magnetic particle may be selected from all magnetic particles, of which surface may be bound with protein G or protein A. For example, the magnetic particles may be magnetic particles having an average particle diameter of about 500 nm to about 10 μm, about 550 nm to about 9 μm, about 600 nm to about 8 μm, about 650 nm to about 7 μm, and about 600 nm to about 6 μm, but are not limited thereto.

When the magnetic particles have a small particle size, individual particles have a single magnetic domain, and therefore, exhibit superparamagnetism having magnetic properties only in the presence of an external magnetic field. Magnetic particles exhibiting superparamagnetism may be simply and quickly separated by applying an external magnetic field. Since separation of magnetic particles by application of a magnetic field is not affected by surrounding environments, such as pH, temperature, ions, etc., they are excellent in terms of stability and sensitivity.

In the composition according to an aspect, the magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof, may serve as a feeder cell which has been traditionally used in culturing NK cells to enhance proliferation of NK cells and to expand NK cells. A feeder cell which has been traditionally used, for example, K562, is a cancer cell line, and the cell adversely affects the human body, and thus is difficult to use clinically. Therefore, when the magnetic particles are used instead of feeder cells, clinically safe NK cells may be cultured to be used in cell therapy products.

In the composition according to one aspect, the culturing may be for proliferation, activation, or expansion of NK cells. The proliferation of NK cells means an increase in the number of cells, and may be used interchangeably with growth. The activation of NK cells may mean that the aforementioned NK cells perform their functions. The activation of NK cells may be confirmed through an increase in the aggregation of NK cells or PBMCs including the same.

In the composition according to one aspect, the culturing may be for inducing a dominant environment for NK cells in PBMCs. Induction of the dominant environment for NK cells may mean an increase in a percentage of NK cells and the number of NK cells in the cultured PBMCs, when NK cells are cultured by the composition, as compared with those cultured without the composition.

In the composition according to one aspect, the culturing may be for inhibiting NK cells.

In the composition according to one aspect, the culturing may be for inducing interferon secretion of NK cells. NK cells cultured by the composition may be NK cells with improved function of secreting interferon, for example, interferon gamma, as compared with those cultured without the composition.

In the composition according to one aspect, the culturing may be for improving cytotoxicity and cell killing ability of NK cells. The NK cells cultured by the composition may be usefully applied to the treatment of diseases, for example, cancer, because they have a characteristic of improved cell killing ability.

In the composition according to one aspect, the culturing may be for changing receptor expression of NK cells. In one embodiment, the culturing may be for increasing or decreasing expression of activating receptors of NK cells. In another embodiment, the culturing may be for increasing or decreasing expression of inhibitory receptors of NK cells.

In the present disclosure, the surface antigen characteristic has the same meaning as the immunological characteristic, and may be identified by observing a cell surface marker (e.g., staining cells with a tissue-specific or cell-marker-specific antibody) using a technique such as flow cytometry or immunocytochemistry, or using an optical microscope or a confocal microscope, or by measuring changes in gene expression using a technique well known in the art, such as polymerase chain reaction (PCR) or gene-expression profiling.

The “positive or +”, with respect to a cell marker, may mean that the marker is present in a large amount or at a high concentration, as compared with that in other cells as a reference. Any marker is present inside or on the surface of a cell, and therefore, when a cell may be distinguished from one or more other cell types by using the marker, the cell may be positive for the marker. Further, the “positive” may mean that cells have signals of higher intensity than a background intensity, for example, cells have the marker in an amount enough to be detectable in a cell-measuring device. For example, cells may be detectably labeled with CD56-specific antibodies, and when signals from these antibodies are detectably stronger than those of a control (e.g., background intensity), the cells may be “positive for CD56” or “CD56+”. The term “negative or −” may mean that although antibodies specific to a particular cell surface marker are used, the marker cannot be detected, as compared with the background intensity. For example, when a cell cannot be detectably labeled with a CD3-specific antibody, the cells may be “negative for CD3” or “CD3−”.

In the composition according to one aspect, the NK cells may be in a form of being included in peripheral blood mononuclear cells (PBMCs). The PBMCs may be autologous or allogeneic PBMCs, and may be PBMCs derived from a healthy individual or a patient.

The medium refers to a material capable of supporting growth and survival of cells in vitro. The medium is not particularly limited, as long as it may be used in cell culture, and the medium may include, for example, one or more selected from the group consisting of Dulbecco's Modified Eagle's Medium (DMEM), Minimal Essential Medium (MEM), Basal Medium Eagle (BME), RPMI 1640, F-10, F-12, DMEM/F12, Minimal Essential Medium-α (MEM-α), Glasgow's Minimal Essential Medium (G-MEM), Iscove's Modified Dulbecco's Medium (IMDM), MacCoy's 5A medium, AmnioMax complete medium, AminoMax II complete medium, Endothelial Basal Medium (EBM) medium, and Chang's Medium.

Another aspect provides a kit for culturing NK cells, the kit including the composition for culturing NK cells according to one aspect and a culture plate.

The composition, the NK cells, and the culture are the same as described above.

The culture plate refers to a cell culture vessel, and includes a cell culture vessel, regardless of a material, size, and shape of the culture plate.

Still another aspect provides a method of culturing NK cells using the composition for culturing NK cells.

The method of culturing NK cells includes culturing NK cells in a medium including the composition for culturing NK cells, the composition including magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof.

The method according to an aspect may further include obtaining PBMCs, before the culturing.

In addition, the method may further include isolating NK cells from the obtained PBMCs.

A method of isolating blood, a method of isolating and obtaining PBMCs therefrom, and a method of isolating NK cells therefrom may be performed by a known method of using specific antibodies, etc.

The method according to an aspect may further include removing the magnetic particles from the medium, after the culturing.

In the method according to an aspect, the culturing may be performed for about 6 days to about 21 days, about 6 days to about 20 days, about 6 days to about 18 days, or about 6 days to about 15 days.

In the method according to an aspect, the culturing may be for proliferating or activating or expanding NK cells.

In the method according to an aspect, the culturing may be for inhibiting NK cells.

Still another aspect provides NK cells prepared by the method of culturing NK cells.

Still another aspect provides a composition for preventing or treating cancer, the composition including the NK cells prepared by the method of culturing NK cells.

The cancer may be solid cancer, lung cancer, liver cancer, breast cancer, uterine cancer, blood cancer, etc., but is not limited thereto. It has been reported that these NK cells are closely related to development of diseases, such as lung cancer (Carrega P, et al., Cancer, 112, 863-875, 2008), liver cancer (Jinushi M, et al., J Hepatol., 43, 1013-1020, 2005), breast cancer (Bauernhofer T, et al., Eur J Immunol., 33, 119-124, 2003), uterine cancer (Mocchegiani E., et al., Br j Cancer., 79, 244-250, 1999), blood cancer (Tajima F., et al, Lekemia, 10, 478-482, 1996), etc.

The composition may include a pharmaceutically acceptable carrier. In the composition, the “acceptable carrier” refers to a material, generally, an inert material used in combination with an active ingredient to aid application of the active ingredient. The carrier may be an excipient, a disintegrant, a binder, a lubricant, a diluent, or a combination thereof. The excipient may be microcrystalline cellulose, lactose, low-substituted hydroxycellulose, or a combination thereof. The disintegrant may be sodium starch glycolate, anhydrous dibasic calcium phosphate, or a combination thereof. The binder may be polyvinylpyrrolidone, low-substituted hydroxypropyl cellulose, hydroxypropyl cellulose, or a combination thereof. The lubricant may be magnesium stearate, silicon dioxide, talc, or a combination thereof.

Still another aspect provides a method of treating cancer, the method including administering, to an individual, a therapeutically or pharmaceutically effective amount of the NK cells prepared by the method of culturing NK cells.

The term “administering” refers to introduction of a predetermined substance into an individual in any appropriate manner, and with regard to the administration route, the substance may be administered through any general route as long as it may reach a target tissue. The administration route may be intraperitoneal administration, intravenous administration, intramuscular administration, subcutaneous administration, intradermal administration, oral administration, topical administration, intranasal administration, intrapulmonary administration, or rectal administration, but is not limited thereto. In addition, administration may be performed by any apparatus capable of moving to a target cell. An administration dose may be appropriately selected according to the type of cancer, the administration route, a patient's age and gender, and severity of a disease, but for an adult, it may be administered at a dose of about 1×10⁶ cells to about 1×10″ cells on average.

The “therapeutically effective amount” means an amount sufficient to exhibit a therapeutic effect when administered to an individual or a cell in need of treatment. The “treatment” means treating a disease or medical condition in an individual, for example, a mammal, including a human, and the treatment includes: (a) prevention of generation of a disease or medical symptoms, i.e., preventive treatment of a patient; (b) relief of a disease or medical symptoms, i.e., removal or recovery of a disease or medical symptoms in a patient; (c) suppression of a disease or medical symptoms, i.e., slowing or stopping progression of a disease or medical symptoms in an individual; or (d) alleviation of a disease or medical symptoms in an individual.

### Advantageous Effects of Disclosure

According to a composition for culturing natural killer cells according to aspect, and a method of culturing natural killer cells using the same, when natural killer cells are cultured in peripheral blood mononuclear cells, they are cultured in a medium including the composition for culturing natural killer cells, the composition including magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof, thereby proliferating natural killer cells in a large quantity and promoting activation, inhibition, or expansion of natural killer cells. Accordingly, the natural killer cells cultured using the same may be usefully applied to immune cell therapy products. Further, the magnetic particles may be easily separated from the medium, which is a simple and economical manner. Since the safe magnetic particles are used, they are excellent in terms of clinical safety.

### BRIEF DESCRIPTION OF DRAWINGS

FIG. 1 shows microscopic images (×40) showing morphology of PBMCs after being cultured for 5 days using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 2 shows results of counting cells using a hemocytometer on days 6 and 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 3 shows a graph of a paired t-test for the results of counting cells using a hemocytometer on days 6 and 12 of culture using soluble IL-15 (A), soluble IL-15 and magnetic particles (B), soluble IL-15 and 4-1BBL_IgG1Fc-bound magnetic particles (C), soluble IL-15 and 4-1BBL_IgG1Fc- and IL-15Rα_IgG1Fc-bound magnetic particles (D);

FIG. 4 shows results of FACS by CD3 and CD56 marker staining on day 0 of culture (A), on day 12 of culture (B), on day 12 of culture using soluble IL-15 (C), on day 12 of culture using soluble IL-15 and magnetic particles bound with no particular molecule (D), on day 12 of culture using soluble IL-15 and 4-1BBL_IgG1Fc-bound magnetic particles (E), and on day 12 of culture using soluble IL-15 and 4-1BBL_IgG1Fc- and IL-15Rα_IgG1Fc-bound magnetic particles (F);

FIG. 5 shows a comparison between 5 donors for FACS results by CD3 and CD56 marker staining on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 6 shows a graph showing results of calculating the number of NK cells in each donor on days 6 and 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 7 shows FACS results by CFSE and 7-AAD staining to evaluate cell killing ability of PBMCs on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 8 shows a graph showing a comparison of cell killing ability of PBMCs between 4 donors on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 9 shows ELISA results of detecting IFN-γ in culture supernatants of PBMCs on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D);

FIG. 10 shows overlay histograms of analyzing NK cell surface receptor expression in PBMCs during culture using (A) soluble IL-15; (B) soluble IL-15 and 4-1BBL_IgG1Fc-bound magnetic particles; or (C) soluble IL-15 and 4-1BBL_IgG1Fc- and IL-15Rα_IgG1Fc-bound magnetic particles;

FIG. 11 shows results of a paired t-test for statistical analysis of NK cell receptor expression in PBMCs during culture using (A) soluble IL-15; (B) soluble IL-15 and 4-1BBL_IgG1Fc-bound magnetic particles; or (C) soluble IL-15 and 4-1BBL_IgG1Fc- and IL-15Rα_IgG1Fc-bound magnetic particles; and

FIG. 12 shows a diagram illustrating a method of culturing NK cells using a composition for culturing NK cells according to an aspect.

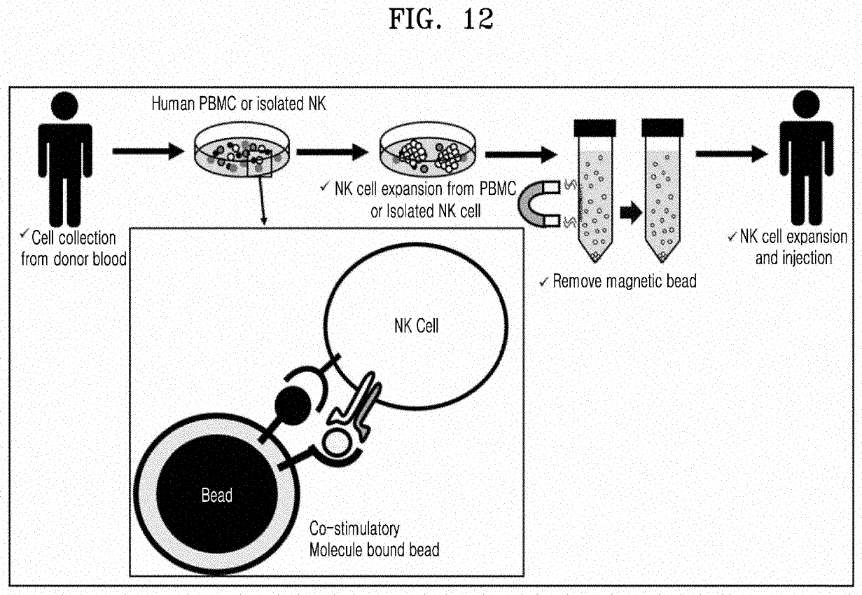

### MODE OF DISCLOSURE

Hereinafter, the present disclosure will be described in more detail with reference to exemplary embodiments. However, these exemplary embodiments are only for illustrating one or more specific embodiments, and the scope of the present disclosure is not limited to these exemplary embodiments.

### Example 1: Culture of Natural Killer (NK) Cells Using Magnetic Particles for Culturing NK Cells

1.1 Preparation of Magnetic Particles for Culturing NK Cells

As magnetic particles which may serve as feed cells during culture of NK cells, 4-1BB ligand- or IL-15Rα-bound magnetic particles were prepared. To bind a specific protein (4-1BB ligand or IL-15Rα) to magnetic particles, magnetic particles coated with protein G were used. Protein G has very high binding affinity to human immunoglobulin. Therefore, when a specific protein fused with human immunoglobulin is used, the specific protein may be bound to magnetic particles through binding between protein G and the immunoglobulin.

In detail, Dynabeads (Thermo Fisher Scientific, Novex) which are magnetic particles coated with protein G were purchased. As the specific proteins, interleukin-15 receptor alpha (IL-15Rα) and 4-1BB ligand (4-1BBL) were used, and an IL-15Rα-fused immunoglobulin-tagged protein (hereinafter, referred to as ‘IL-15Rα_IgG1Fc’, R&D systems, Minneapolis, Minn., USA) and a 4-1BBL-fused immunoglobulin-tagged protein (hereinafter, referred to as ‘4-1BBL_IgG1Fc’, ACRObiosystems, Newark, Del., USA) were purchased.

The immunoglobulin-tagged protein and protein G-coated magnetic particles were allowed to react in a cold room for about 1 hour, and the supernatant was removed from MagneSphere magnetic stand (Promega, Madison, USA) by buffer aspiration, thereby preparing ‘magnetic particles for culturing NK cells’.

1.2 Culture of NK Cells

NK cells were obtained from peripheral blood mononuclear cells (PBMCs) by culturing. PBMCs were obtained from 5 healthy blood donors under the Institutional Review Board (IRB) approval (1044308-201701-BR-005) by College of Medicine, CHA University. Each collected whole blood was diluted with phosphate buffered saline (PBS). PBMCs were isolated from each the blood sample diluted with PBS, and put in a SepMate tube (STEMCELL Technologies, Inc., Vancouver, Canada). PBMC isolation was performed by Ficoll-Hypaque density gradient centrifugation using Histopaque-1077 (Sigma-Aldrich, St. Louis, USA).

Culturing of the obtained PBMCs was performed at a density of 1×10⁶ PBMCs/well in 24 wells. A portion of the medium was changed or added every 2 to 4 days depending on the morphology of PBMCs, and cell counting was performed every 6 days (days 6 and 12). PBMCs were maintained in a basic RPMI-1640 (Hyclone, Logan, USA) supplemented with 10% heat-inactivated FBS (Gibco, Thermo Fisher, USA), 50 μM beta-mercaptoethanol, and 1% penicillin and streptomycin (P/S).

Hereinafter, NK cells were cultured by a method of culturing NK cells in PBMCs using whole PBMCs obtained in Example 1.2, and experimental groups used for experiments were as follows: \textcircled{1} culture in a medium including soluble IL-15Rα or 4-1BBL, \textcircled{2} culture in a medium including the magnetic particles of Example 1.2, and \textcircled{3} culture in a medium including magnetic particles to which specific molecules were not bound.

### Experimental Example 1: Evaluation of NK Cell Proliferation Using Magnetic Particles for Culturing NK Cells

NK cell proliferation efficiency, when the magnetic particles for culturing NK cells prepared in Example 1.1 were used, was evaluated. To measure NK cell proliferation, the three types of experimental groups were subjected to flow cytometry every 6 days (on day 0, day 6, and day 12). PBMC proliferation was measured using a hemocytometer. After PBMC counting, a portion of the sample was used for flow cytometry. PBMC samples were treated and stained with a CD3 PE antibody (Thermo Fisher Scientific) and a CD56 FITC antibody (Thermo Fisher Scientific). Through staining with these two antibodies, populations of NK cells, NKT cells, T cells, B cells, and monocytes in the obtained PBMCs may be distinguished.

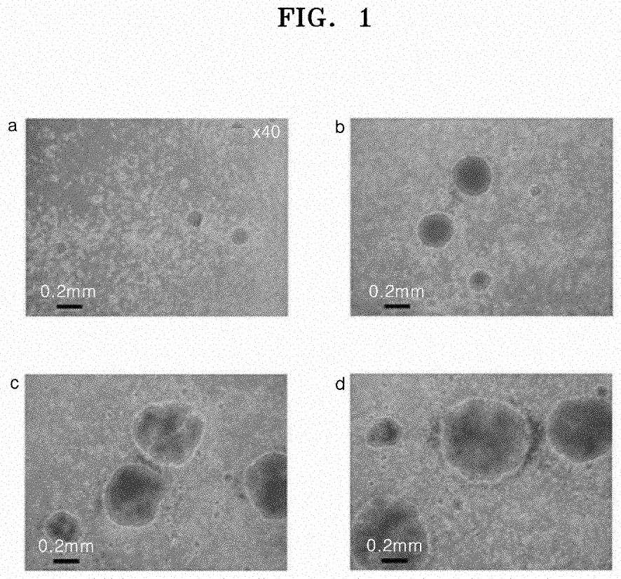

FIG. 1 shows microscopic images (×40) showing morphology of PBMCs after being cultured for 5 days using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D).

As shown in FIG. 1, better aggregation of cells was observed in the PBMC group cultured using magnetic particles for culturing NK cells according to an aspect, to which 4-1BBL or IL-15Rα was bound, as compared with those cultured using soluble IL-15.

It is known that a phenomenon of cluster formation by NK cell aggregation during culture indicates enhancement of NK cell activation. Therefore, when PBMCs were cultured using the 4-1BBL or IL-15Rα-bound magnetic particles according to an aspect, PBMCs well aggregated, indicating NK cell activation.

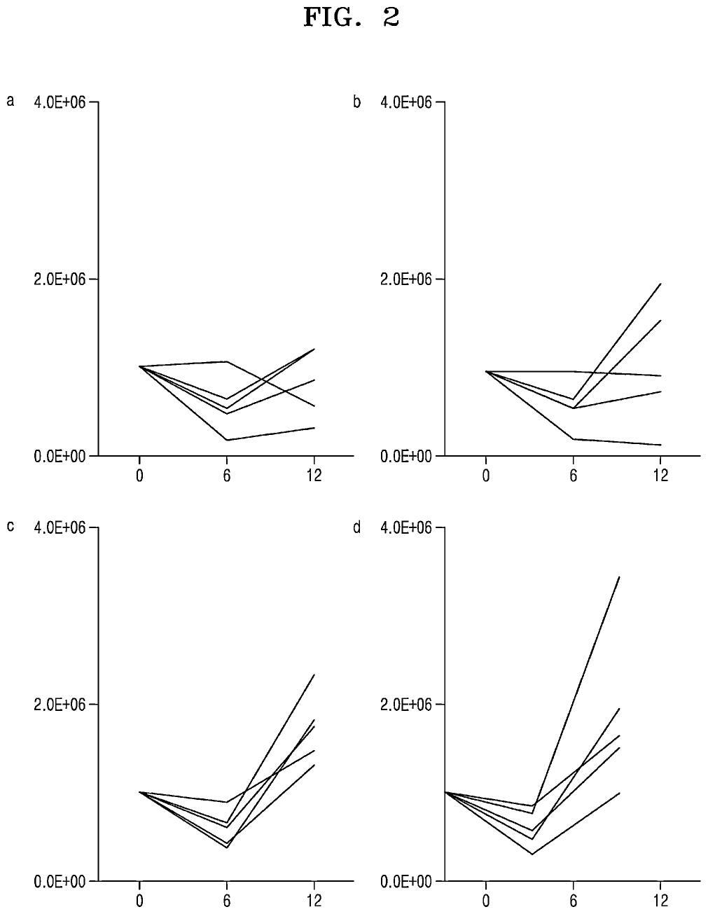

FIG. 2 shows results of counting cells using a hemocytometer on days 6 and 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D).

As shown in FIG. 2, the number of PBMCs in all groups was found to increase on day 12, overall. In particular, it was confirmed that the number of cells significantly increased when PBMCs were cultured using the magnetic particles for culturing NK cells according to an aspect, to which 4-1 BBL and/or IL-15Rα were/was bound.

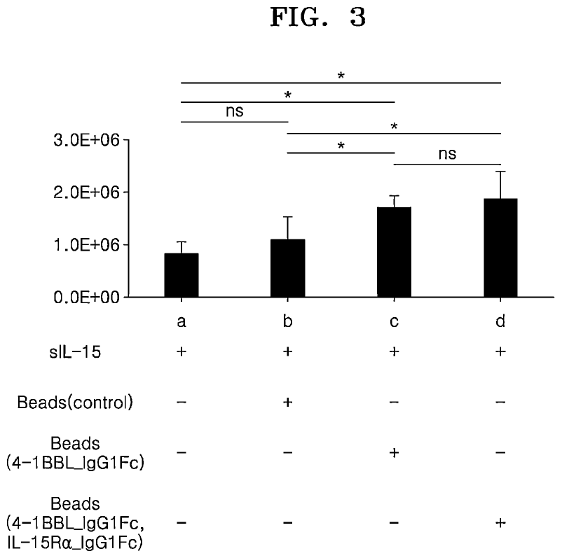

FIG. 3 shows a graph of a paired t-test for the results of counting cells using a hemocytometer on days 6 and 12 of culture using soluble IL-15 (A), soluble IL-15 and magnetic particles (B), soluble IL-15 and 4-1BBL_IgG1Fc-bound magnetic particles (C), soluble IL-15 and 4-1BBL_IgG1Fc- and IL-15Rα_IgG1Fc-bound magnetic particles (D).

As shown in FIG. 3, as the result of comparing the number of PBMCs treated with soluble IL-15 and the experimental group, there was no significant difference in the number of PBMCs treated with soluble IL-15 and magnetic particles. In contrast, the number of cells was found to significantly increase when PBMCs were cultured using the 4-1BBL_IgG1Fc or IL-15Rα_IgG1Fc-bound magnetic particles, and when magnetic particles, to which both 4-1BBL_IgG1Fc and IL-15Rα_IgG1Fc were bound, were used, the greatest increase was observed in the number of NK cells.

### Experimental Example 2: Evaluation of Proportion of NK Cells in PBMCs According to Use of Magnetic Particles for Culturing NK Cells

It was evaluated whether a proportion of NK cells in PBMCs was changed, when the magnetic particles for culturing NK cells prepared in Example 1.1 were used. After PBMCs were stained with each CD marker (CD3, CD56), cell populations were examined by flow cytometry.

In detail, a proportion of NK cells in PBMCs on days 0, 6, and 12 of culture was measured using a CD3 PE antibody (Thermo Fisher Scientific) and a CD56 FITC antibody (BD Pharmingen™), which are NK cell markers, by a flow cytometer, CytoFLEX (Beckman Coulter, Inc., Brea, Calif., USA).

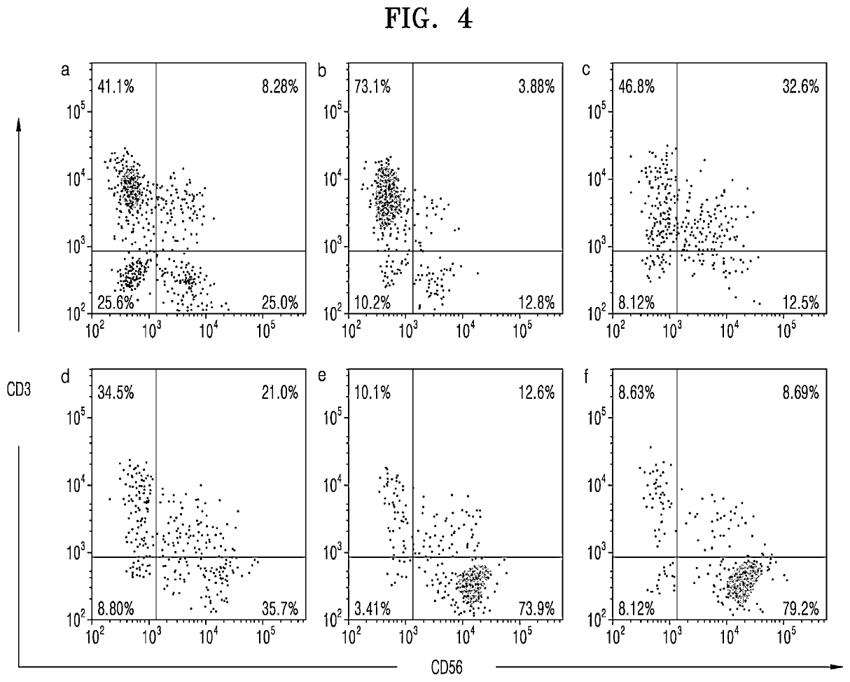

FIG. 4 shows results of FACS by CD3 and CD56 marker staining on day 0 of culture (A), on day 12 of culture (B), on day 12 of culture using soluble IL-15 (C), on day 12 of culture using soluble IL-15 and magnetic particles bound with no particular molecule (D), on day 12 of culture using soluble IL-15 and 4-1BBL_IgG1Fc-bound magnetic particles (E), and on day 12 of culture using soluble IL-15 and 4-1BBL_IgG1Fc- and IL-15Rα_IgG1Fc-bound magnetic particles (F). In the FACS data, the x-axis represents CD56 and the y-axis represents CD3.

As shown in FIG. 4, the average proportion of NK cells before culture (A) was 15.58±4.40% (range, 7.29-32.10). As a result of culturing according to each condition, the proportion of NK cells in FIGS. 4C and 4D was 11.73±0.93% (range, 5.05-18.4) or 19.19±2.35% (range, 6.70-35.70), respectively. In contrast, the proportion of NK cells in PBMCs cultured with the 4-1BBL_IgG1Fc- or 4-1BBL_IgG1Fc-bound magnetic particles (FIGS. 4E and 4F) was 46.74±6.45% (range, 4.53-74.70) and 49.59±6.43% (range, 9.00-79.20), respectively, indicating that expression of NK cell markers was significantly increased (p<0.001).

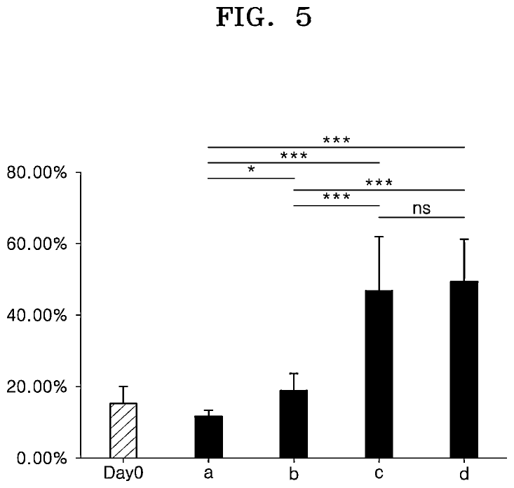

FIG. 5 shows a comparison between 5 donors for FACS results by CD3 and CD56 marker staining on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D). As shown in FIGS. 4 and 5, it was confirmed that the proportion of NK cells in PBMCs was statistically significantly increased in the group cultured with the magnetic particles of one aspect, to which specific molecules were bound.

In addition, to examine the change in the number of NK cells after culture, a percentage of NK cells obtained by antibody staining was multiplied by the number of each PBMC obtained in FIG. 2. Then, the number of NK cells in each donor was plotted according to the experimental conditions.

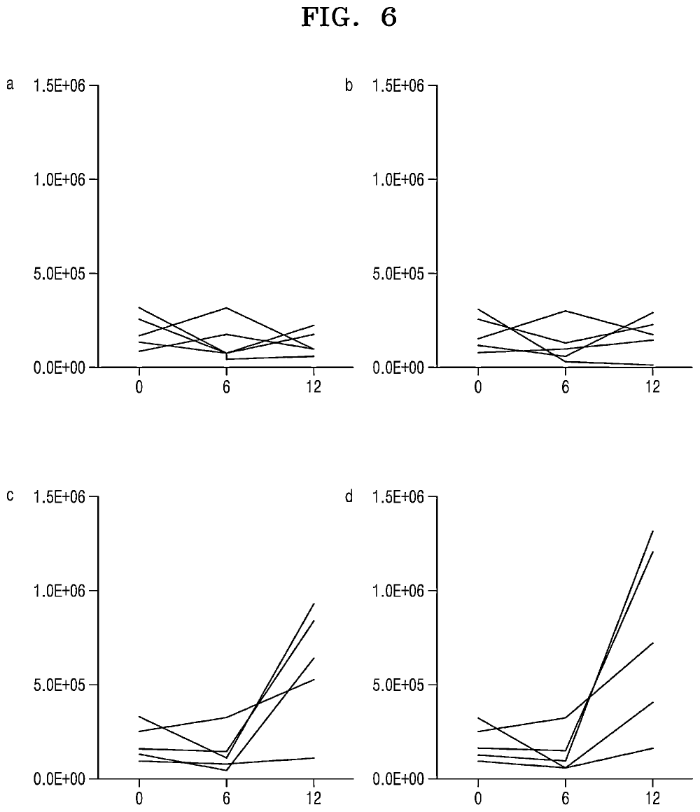

FIG. 6 shows a graph showing results of calculating the number of NK cells in each donor on days 6 and 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D).

As shown in FIG. 6, it was confirmed that the number of NK cells significantly increased, when cultured with magnetic particles to which specific molecules were bound, as compared with the other two controls.

In addition to the percentage data of NK cells (CD3-, CD56+), data on T cells (CD3+, CD56-), NKT cells (CD3+, CD56+), B cells, and monocytes (CD3-, CD56-) were also obtained in the same manner using CD3 and CD56 antibodies. The results of measuring a fold change in the percentage of whole PBMCs including NK cells (CD3-, CD56-) are shown in Table 1 below. Table 1 shows the result of charting the fold change of the increase rate of the whole PBMCs including NK cells, T cells, and NKT cells. Statistical analysis was performed by a paired t-test.

TABLE 1

| | | | | sIL-15 |
|---|---|---|---|---|
| | | | sIL-15 | +Magnetic |
| | | sIL-15 | +Magnetic | particles |
| | sIL-15 | +Magnetic | particles | (4-1BBL, |
| | (Control) | particles | (4-1BBL) | IL-15Rα) |
| Whole | 1 ± 0.21 | 1.32 ± 0.25 | 2.07 ± 0.88 | 2.27 ± 0.88 |
| PBMCs | | | | |
| NK cells | 1 ± 0.25 | 1.42 ± 0.31 | 4.90 ± 2.81* | 6.08 ± 2.20* |
| NKT cells | 1 ± 0.30 | 1.91 ± 1.09 | 1.48 ± 0.30 | 1.97 ± 0.69 |
| T cells | 1 ± 0.46 | 0.93 ± 0.69 | 1.06 ± 0.24 | 1.13 ± 0.42 |

(*P < 0.05). Both the percentage and the number of NK cells significantly increased, when cultured with magnetic particles to which specific molecules were bound, indicating that, when PBMCs are cultured with magnetic particles to which specific molecules were bound, the environment inside PBMCs is induced to the NK cell dominant environment.

### Experimental Example 3: Evaluation of PBMC-Mediated Cell Killing Ability According to Use of Magnetic Particles for Culturing NK Cells

To evaluate improvement of immune cell function of NK cells, in addition to the increase in the number of NK cells in PBMCs, by using the magnetic particles for culturing NK cells prepared in Example 1.1, PBMC-mediated cell killing ability against K562 was evaluated.

In detail, a leukemia cell line K562 was treated with PBMCs cultured for 12 days as described above. First, CFSE staining was performed to distinguish the target cell K562 from the effector cell PBMC in flow cytometry. Only the effector cells (NK cells) were gated and cultured with K562 for 4 hours, and then, 7-AAD staining was performed to detect dead K562 cells. Here, the co-culture was performed at a ratio of E:T=10:1. By detecting values of the 7-AAD staining, it was examined how many % of the target cells were killed. The dead cells were selected based on a K562 dot plot.

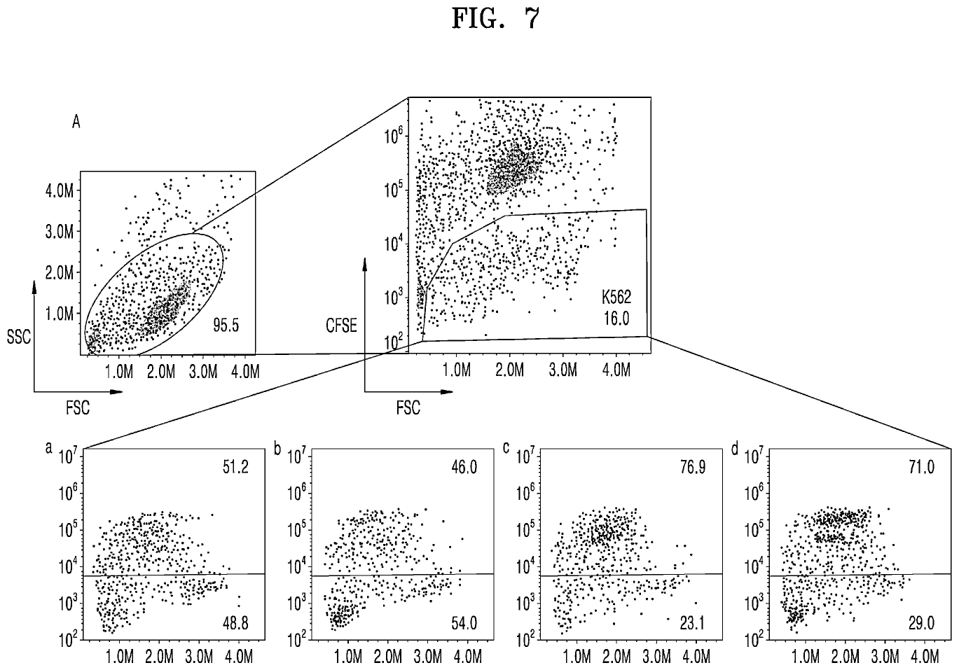

FIG. 7 shows FACS results by CFSE and 7-AAD staining to evaluate cell killing ability of PBMCs on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D). In the FACS data, the y-axis represents 7-AAD, and the stained cells on the line were actually dead cells. CSFE is used for the purpose of distinguishing NK cells from K562 cells, and is widely stained in the cytoplasm. 7-AAD is used for the purpose of staining dead cells, and when DNA break occurs, it binds to a base, and as a result, staining occurs.

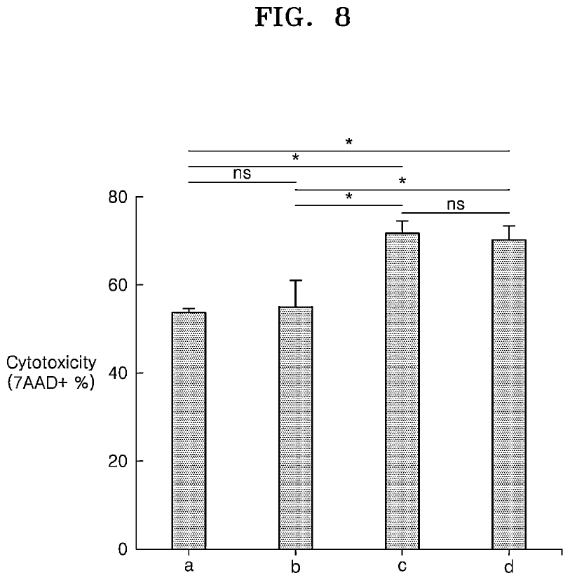

FIG. 8 shows a graph showing a comparison of cell killing ability of PBMCs between 4 donors on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D). The results are results of statistical analysis by a paired t-test for the flow cytometry results, and data were expressed as mean±standard deviation (* P<0.05).

As shown in FIGS. 7 and 8, it was confirmed that the cell killing function of immune cells against K562 was increased, when treated with magnetic particles to which specific molecules were bound.

Therefore, since the cell killing ability against K562 is improved in the PBMC group cultured with magnetic beads according to an aspect, not only the number of cells but also the cytotoxic function is improved.

### Experimental Example 4: Evaluation of Interferon Secretion of PBMCs According to Use of Magnetic Beads for Culturing NK Cells

To evaluate whether interferon secretion of PBMCs is improved by using the magnetic particles for culturing NK cells prepared in Example 1.1, an experiment was performed to quantify the amount of IFN-γ which is closely related to the cytotoxic function and highly secreted from activated NK cells.

The supernatant cultured for 12 days under each experimental condition as described above was used. IFN-γ was detected by enzyme-linked immune-specific assay (ELISA) using an antibody-coated IFN-γ-capture plate.

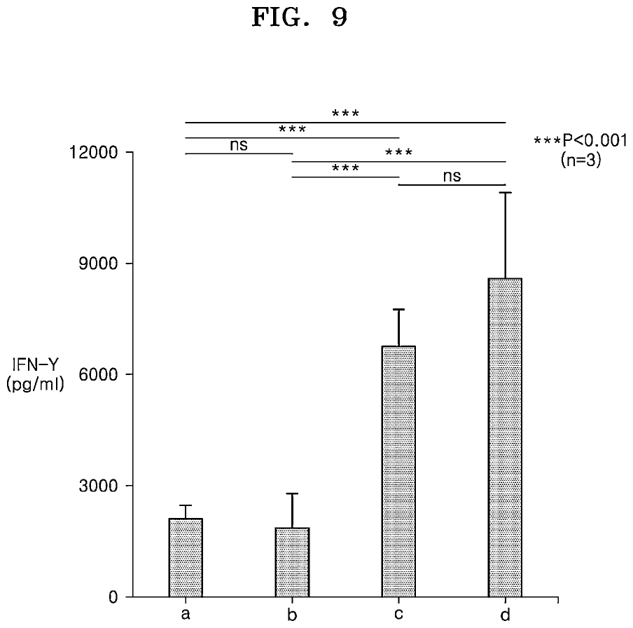

FIG. 9 shows ELISA results of detecting IFN-γ in culture supernatants of PBMCs on day 12 of culture using soluble IL-15 (A), soluble IL-15 and magnetic particles (B), soluble IL-15 and 4-1BBL_IgG1Fc-bound magnetic particles (C), or soluble IL-15 and 4-1BBL_IgG1Fc- and IL-15Rα_IgG1Fc-bound magnetic particles (D). Data were expressed as mean±standard deviation (***P<0.001).

As shown in FIG. 9, IFN-γ was detected significantly high in the culture supernatant of the PBMC group cultured with magnetic particles to which soluble IL-15_IgGFc, 4-1BB_IgGFc and IL-15Rα were bound.

### Experimental Example 5: Evaluation of Change in NK Cell Receptor Expression According to Use of Magnetic Particles for Culturing NK Cells

When the magnetic particles for culturing NK cells prepared in Example 1.1 were used, the proliferation and percentage of NK cells and PBMC-mediated cytotoxicity were increased, and therefore, changes in the NK cell receptor expression were analyzed.

In detail, samples obtained from 3 donors according to each group described above were cultured for 12 days, and then 6 types of receptor molecules were identified using antibodies against the receptor molecules. The used receptor molecules were DNAM1, CD27, NKG2A, NKG2D, CD69, and CD16. Experimental groups used are as follows: (A) soluble IL-15; (B) culture using soluble IL-15 and 4-1BBL_IgG1Fc-bound magnetic particles; and (C) culture using soluble IL-15 and 4-1BBL_IgG1Fc- and IL-15Rα_IgG1Fc-bound magnetic particles.

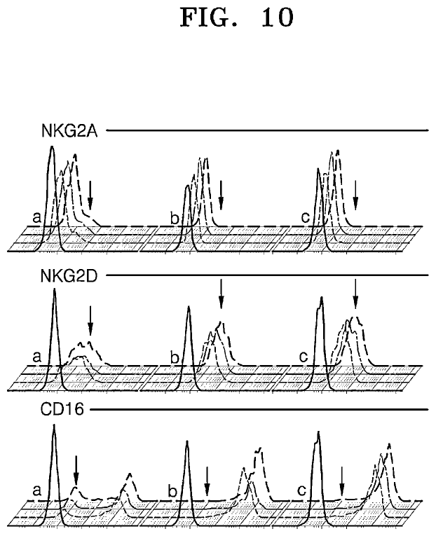

FIG. 10 shows overlay histograms of analyzing NK cell surface receptor expression in PBMCs during culture using (A) soluble IL-15; (B) soluble IL-15 and 4-1BBL_IgG1Fc-bound magnetic particles; or (C) soluble IL-15 and 4-1BBL_IgG1Fc- and IL-15Rα_IgG1Fc-bound magnetic particles.

In addition, the data on the NK cell receptor expression were prepared in a table and statistically processed. Paired t-tests were performed using data of the NK cell receptor expression rate of each donor.

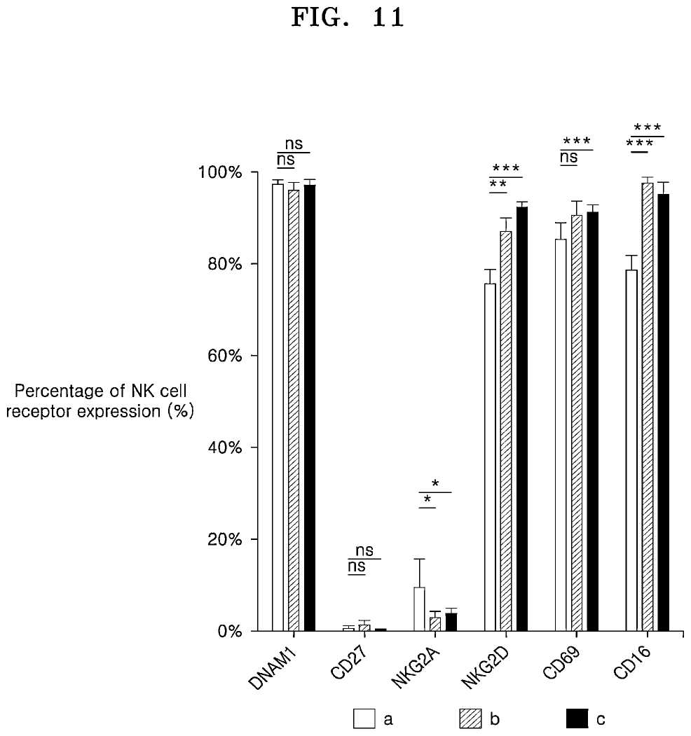

FIG. 11 shows results of a paired t-test for statistical analysis of NK cell receptor expression in PBMCs during culture using (A) soluble IL-15; (B) soluble IL-15 and 4-1BBL_IgG1Fc-bound magnetic particles; or (C) soluble IL-15 and 4-1BBL_IgG1Fc- and IL-15Rα_IgG1Fc-bound magnetic particles. Data were expressed as mean±standard deviation. (*P<0.05, **P<0.005, ***P<0.001)

As shown in FIGS. 10 and 11, NKG2A which is an NK cell inhibitory receptor was slightly expressed in the soluble IL-15 group (A), but decreased in the group using magnetic particles according to an aspect, to which the specific molecules were bound. In contrast, expression percentages of NKG2D, CD69, and CD16 which are activating receptors were found to increase in the group using the magnetic particles according to one aspect.

Therefore, it was confirmed that, when NK cells are cultured using the magnetic particles according to an aspect, inhibitory receptors were decreased and activating receptors were increased in NK cells by culturing with the magnetic particles.

1. A composition for culturing natural killer cells, the composition comprising magnetic particles of which at least one surface is bound with an activating receptor ligand, an inhibitory receptor ligand, a costimulatory receptor ligand, a cytokine, a cytokine receptor, an immune checkpoint ligand, a blocking antibody, or a combination thereof.

2. The composition of claim 1, wherein the activating receptor ligand is one or more selected from the group consisting of an NCR family ligand, an NKG2 family ligand, a KIR family ligand, BAG6, AICL, MICA, MICB, CADM1, IgG, CD48, NTB-A/SLAMF6, CD70, CD155, CD319, C8, C9, and CS1.

3. The composition of claim 1, wherein the inhibitory receptor ligand is one or more selected from the group consisting of HLA-A, HLA-B, HLA-BW4, HLA-C1, HLA-C2, HLA-E, HLA-G, CD112/Nectin-2, CD112/Nectin-3, cadherin, collagen, OCIL, and CLEC2D.

4. The composition of claim 1, wherein the costimulatory receptor ligand is one or more selected from the group consisting of a TNF family ligand, a TLR family ligand, a 4-1BB ligand, CD28 ligand, NTBA, TLRL, PVR/Nectin-2, and PVR.

5. The composition of claim 1, wherein the cytokine is one or more selected from the group consisting of IFN-α, IFN-β, IFN-γ, IL-1, IL-2, IL-3, IL-4, IL-6, IL-10, IL-12, IL-15, IL-17, IL-18, IL-21, and IL-27.

6. The composition of claim 1, wherein the cytokine receptor is IL-2Rα, IL-15Rα, or a combination thereof.

7. The composition of claim 1, wherein the immune checkpoint ligand is one or more selected from the group consisting of B7 family, galectin family, PVR family, PD-L1, a BTLA-4 ligand, a CTLA-4 ligand (CD80), a Tim-3 ligand, and a TIGIT ligand.

8. The composition of claim 1, wherein the blocking antibody is one or more selected from the group consisting of anti-KIR2DL1 monoclonal antibody (mAb), anti-KIR2DL2 mAb, anti-KIR2DL3 mAb, anti-KIR2DL5A mAb, anti-KIR2DL5B mAb, anti-KIR3DL1 mAb, anti-KIR3DL2 mAb, anti-KIR2DL4 mAb, anti-CD94/NKG2A mAb, anti-CD94/NKG2B mAb, anti-CD96 mAb, anti-CEACAM-1 mAb, anti-ILT2/LILRB mAb, anti-KLRG1 mAb, anti-LAIR1 mAb, anti-NKRP1A mAb, anti-Siglec3 mAb, anti-Siglec7 mAb, and anti-Siglec9 mAb.

9. The composition of claim 1, wherein at least one surface of the magnetic particles is coated with protein G or protein A.

10. The composition of claim 1, wherein the activating receptor ligand, the inhibitory receptor ligand, the costimulatory receptor ligand, the cytokine, the cytokine receptor, the immune checkpoint ligand, and the blocking antibody is in a fusion form with a human immunoglobulin.

11. The composition of claim 10, wherein the human immunoglobulin is human immunoglobulin G.

12. The composition of claim 1, wherein the magnetic particles have a size of 500 nm to 10 μm.

13. The composition of claim 1, wherein the culturing is for proliferating or activating natural killer cells.

14. The composition of claim 1, wherein the natural killer cells are comprised in peripheral blood mononuclear cells (PBMCs).

15. A method of culturing natural killer cells, the method comprising culturing natural killer cells in a medium comprising a composition for culturing natural killer cells, the composition comprising magnetic particles, of which at least one surface is bound with an activating receptor ligand, an inhibitory receptor ligand, a costimulatory receptor ligand, a cytokine, a cytokine receptor, an immune checkpoint ligand, a blocking antibody, or a combination thereof.

16. The method of claim 15, further comprising obtaining peripheral blood mononuclear cells, before the culturing.

17. The method of claim 15, further comprising removing the magnetic particles from the medium, after the culturing.

18. The method of claim 15, wherein the culturing is performed for 6 days to 21 days.
